(19) United States
(12) Patent Application Publication
(10) Pub. No.: US 2026/0279173 A1
(43) Pub. Date: Sep. 17, 2026

(54) METHODS AND SYSTEMS FOR IMPARTING TACTILE SENSATIONS HAVING ACOUSTIC TRAITSTO TACTILE REGIONS OF A USER’S HAND OR HANDS

(71) Applicants: UNIVERSITÉ LAVAL, Québec (CA); ECOLE DE TECHNOLOGIE SUPERIEURE, Montréal (CA)

(72) Inventors: Andréanne SHARP, Québec (CA); Éliane LEPROHON, Québec (CA); Jérémie VOIX, Montréal (CA); Aidin DELNAVAZ, Montréal (CA); Valentin PINTAT, Montréal (CA)

(21) Appl. No.: 19/471,457

(22) PCT Filed: Apr. 4, 2024
(86) PCT No.: PCT/CA2024/050437

§ 371 Date: Oct. 1, 2025

Related U.S. Application Data
(60) Provisional application No. 63/494,295, filed on Apr. 5, 2023.

(51) Int. Cl. G08B 6/00; H04R 1/02; H04R 17/00

(52) U.S. Cl. CPC G08B 6/00; H04R 1/025; H04R 17/00; H04R 2201/023

(57) ABSTRACT

In accordance with one aspect, a plurality of transducers mechanically coupled to different tactile regions of at least one hand of a user are driven via different channel signals, the different channel signals having acoustic traits evolving independently from the acoustic traits of the other channel signals. In accordance with another aspect, transducers mechanically coupled to tactile regions of the hand of a user can be piezoelectric transducers.

### BACKGROUND

This specification pertains to methods and systems for imparting tactile sensations, often referred to as “haptics”. While known methods and systems are satisfactory to a certain degree, there remains much room for improvement. Indeed, commercially available haptics at the time of filing this specification were typically limited to continuous vibrations which may or may not vary in intensity, or a limited set of haptic effects such as a snapping or clicking sensation.

### SUMMARY

There are various reasons why one would want to be able to provide tactile sensations (e.g. vibrations) having acoustic traits (e.g. intensity, frequency, sharpness, and/or temporal acoustic traits) to regions of the human body having tactile sensitivity. For instance, musicians may be enabled to more easily coordinate with other musicians, a person who has hearing-related issues, such as being hard of hearing or deaf, may be able to “feel” an acoustic experience to a certain degree, or one may want to amplify an audio, video, or audiovisual experience, such as for example a virtual reality experience, with tactile sensations having acoustic traits. One challenge in achieving this is that the human ear is typically sensitive to a frequency band of between 20 and 20 000 Hz, whereas our tactile (skin) receptors are typically sensitive to frequencies between 2 and 1000 Hz. Accordingly, while there is some degree of overlap, there is a significant frequency band discrepancy between these two sensory systems of the human body. Another challenge in achieving this is to find a way to impart vibrations having a sufficient amplitude in terms of force imparted onto associated sensory receptors of the skin, at different frequencies within the target frequency bandwidth, for the sensory receptors of the user's skin to “feel” the tactile effects to a suitable degree of amplitude.

It was found that there are at least two ways to address the first of these challenges. A first one is to limit the tactile feedback to the frequencies which are shared between the two frequency bands, e.g. to a frequency band of between 20 and 1000 Hz. This approach can translate acoustic frequencies directly into tactile vibrations, but has the inconvenience of being applicable only for a relatively small portion of the acoustic frequency band, potentially limiting the richness of the experience. The second one of these approaches is to perform a frequency shift, i.e. to execute a frequency transformation function on the acoustic signal to translate the acoustic frequencies into tactile frequencies. For instance, a vibration at 20 000 Hz in the audio signal can be translated into a vibration at 1000 Hz, whereas a vibration at 20 Hz can be translated into a vibration at 2 Hz, and intermediary vibration frequencies can be translated in a similar, linear or logarithmic, manner. This second approach, while in appearance potentially counter-intuitive, has the potential advantage of entirely representing the acoustic frequency band as tactile vibrations. Moreover, another factor is to be taken into consideration. Indeed, the human brain has demonstrated an impressive amount of plasticity in its functionalities over a wide range of scientific experiments, and when taking this latter factor into account, it is not unlikely that the brain, provided with tactile feedback in the 2-1000 Hz frequency range, may establish similarities in the acoustic traits conveyed within such tactile sensations, and acoustic traits known to its acoustic interpretation system, and provide some form of transfer function to convert this tactile feedback to a certain extent in a manner to map it onto its existing acoustic processes, potentially providing the user with an acoustic experience to at least a certain degree.

To address the second of these challenges, one may take into account the tactile sensory response of different portions of the human body. Indeed, the nervous system allocated brain power according to need, and some regions of the body requiring high levels of conscious dexterity have larger representations in the brain. The hands and tongue are examples of such regions. Somewhat accordingly, tactile sensitivity varies across the skin, where finger tips and facial skin are particularly receptive to the touch. In this context, targeting the human hand(s), in particular, may be particularly promising from the point of view of artificially providing tactile vibrations which satisfactory “connect” with the tactile receptors of the skin.

This insight leads to considering the questions of what type of electromechanical arrangement and configuration is best adapted to conveying tactile vibrations having acoustic traits onto portions of a person's hand and of which processes can be used to operate such electromechanical arrangements in a manner to reap the full potential of such new technology.

In accordance with one aspect, there is provided a process by which different channel signals are used to drive different transducers mechanically coupled to at least one hand of a user, with the different transducers being positioned at different tactile regions of the user's hand or hands, such as on different hands, on different fingers of the same hand, on one or more finger and a palm of a given hand, etc. Here, mechanically coupling refers to the fact that movement such as vibrations generated by the transducer can be suitably conveyed to the corresponding tactile region of the user's hand, which can be achieved by holding the transducers and any intervening material against the tactile region. The different channel signals can all have one or more of intensity, frequency, and temporal acoustic traits. The different channel signals can be different, independent, signals in that the acoustic traits of each channel signal can be different from the acoustic traits of some or all of the other channel signals at a given moment in time, and evolve independently from one another. For instance, the different channel signals can be mapped to different frequency bands of a source audio signal, or different channel signals can be mapped to different instruments collaborating to play an audio harmony.

In accordance with the latter aspect, there is provided, for instance: a method of imparting tactile sensations, the method comprising: providing a plurality of transducers mechanically coupled to at least one hand of a user, different ones of the plurality of transducers being mechanically coupled to different tactile regions of the at least one hand of the user; driving a first one of the plurality of transducers via a first channel signal to impart tactile sensations to a first one of the tactile regions, the first channel signal having at least one of intensity, frequency, and temporal acoustic traits; and driving a second one of the plurality of transducers via a second channel signal to impart tactile sensations to a second one of the tactile regions, the second channel signal having at least one of intensity, frequency and temporal acoustic traits; the acoustic traits of the second channel signal being different from the acoustic traits of the first channel signal.

Still in accordance with the latter aspect, there is provided, for instance: a method of converting a source audio signal in the audio frequency band of between 20 and 20 000 Hz into a plurality of channel signals each in the tactile frequency band of between 2 and 1000 Hz, the method comprising: splitting the audio frequency band of the source audio signal into partial audio frequency bands; frequency shifting at least portions of the source audio signal from the audio frequency band of between 20 and 20 000 Hz into the tactile frequency band of between 2 and 1000 Hz; frequency scaling the partial audio frequency bands; and outputting the plurality of channel signals.

In accordance with a second aspect, there is provided an electromechanical arrangement which is adapted to conveying tactile sensations having acoustic traits onto tactile regions of a person's hand or hands. More specifically, the use of traditional acoustic transducer arrangements based on permanent magnet, voice coil, and diaphragm arrangements, may be at the source of some inconveniences and not be suitable in all applications. For instance, ferromagnetic materials such as used in traditional acoustic transducer arrangements are not suitable for use in a magnetic resonance imaging (MRI) context, and may not be suitable to stimulate different tactile regions of a person's hand or hands with tactile sensations having acoustic traits while imaging the reaction of that person's brain using MRI. Accordingly, it was found that, at least in some applications, such as MRI contexts, using a piezoelectric transducer could be preferable to using a traditional acoustic transducer arrangement for such purposes.

In accordance with the latter aspect, there is provided, for instance: a system for imparting tactile sensations to different tactile regions of a hand of a user, the system comprising: a garment configured to be worn by a hand of a user; and a plurality of piezoelectric transducers secured to the garment at different locations of the garment, the plurality of piezoelectric transducers being mechanically coupled to the different tactile regions of the hand of the user when the garment is worn by the hand of the user, each piezoelectric transducer being drivable to impart tactile sensations to a corresponding one of the tactile regions by a signal having at least one of intensity, frequency and temporal acoustic traits.

Moreover, still in accordance with the latter aspect, there is provided, for instance: a method of performing magnetic resonance imaging (MRI) comprising: providing a plurality of transducers mechanically coupled to different tactile regions of at least one hand of the user; positioning a head of the user in a MRI system; and driving the transducers in a manner to impart tactile sensations to the different tactile regions while operating the MRI system to acquire imaging data from the head of the user.

Many further features and combinations thereof concerning the present improvements will appear to those skilled in the art following a reading of the instant disclosure.

### DESCRIPTION OF THE FIGURES

In the figures,

FIG. 1 is a view of an example of a system for imparting tactile sensations to different tactile regions of a hand of a user;

FIG. 2 is a schematic view illustrating a first way of providing different channel signals which can be used to drive different transducers of a system for imparting tactile sensations, including splitting the frequency band of a source audio signal into a number of partial audio frequency bands, and shifting and scaling the partial signals associated to the partial audio frequency bands to partial tactile frequency bands, in a linear manner;

FIG. 3 is a schematic view illustrating a second way of providing different channel signals which can be used to drive different transducers of a system for imparting tactile sensations, including splitting a portion of the frequency band of a source audio signal into a number of partial audio frequency bands, and shifting and scaling the partial signals associated to the partial audio frequency bands to partial tactile frequency bands, in a logarithmic manner;

FIG. 4 is a schematic view illustrating a third way of providing different channel signals which can be used to drive different transducers of a system for imparting tactile sensations, including splitting a portion of the frequency band of a source audio signal into a number of partial audio frequency bands, and shifting and scaling the partial signals associated to the partial audio frequency bands to a same tactile frequency band;

FIG. 5 is a schematic view illustrating a third way of providing different channel signals which can be used to drive different transducers of a system for imparting tactile sensations, including phase shifting partial signals associated to different instruments in audio frequency bands to tactile frequency bands;

FIG. 6 is a flow chart presenting a process of imparting tactile sensations to different ones of a plurality of tactile regions of at least one hand of a user;

FIG. 7 is a schematic view illustrating a set of example tactile regions of hands of a user;

FIGS. 8A and 8B are views of an experimental setup of a system using audio transducers;

FIG. 8C is a graph presenting experimental results of the setup of FIGS. 8A and 8B;

FIG. 9 is a degrees of freedom model of the experimental setup of FIGS. 8A and 8B;

FIG. 10 visually represents a process of performing magnetic resonance imaging while imparting tactile sensations;

FIG. 11 shows an example of a system for imparting tactile sensations;

FIG. 12 presents force transfer response as a function of stiffness, for different frequencies;

FIG. 13 presents an example of a mechanical arrangement for mechanically coupling a piezoelectric transducer to a tactile region;

FIG. 14 presents a mechanical arrangement combining two piezoelectric transducers;

FIG. 15 presents a mechanical arrangement combining two piezoelectric transducers;

FIG. 16 is a graph presenting experimentally acquired sensation thresholds for various mechanical arrangements;

FIG. 17 is a graph presenting experimentally acquired sensation thresholds for various mechanical arrangements; and

FIG. 18 is a block diagram of an example computer.

### DETAILED DESCRIPTION

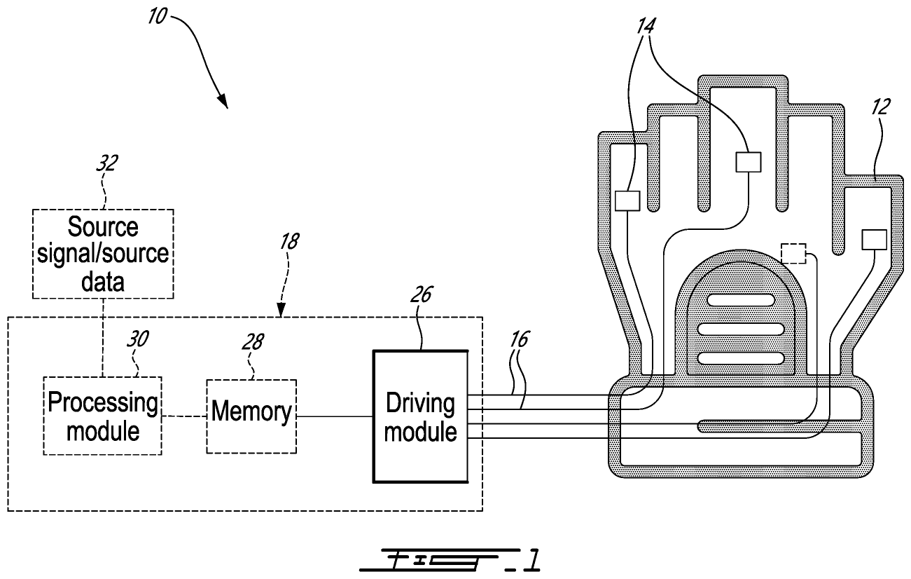

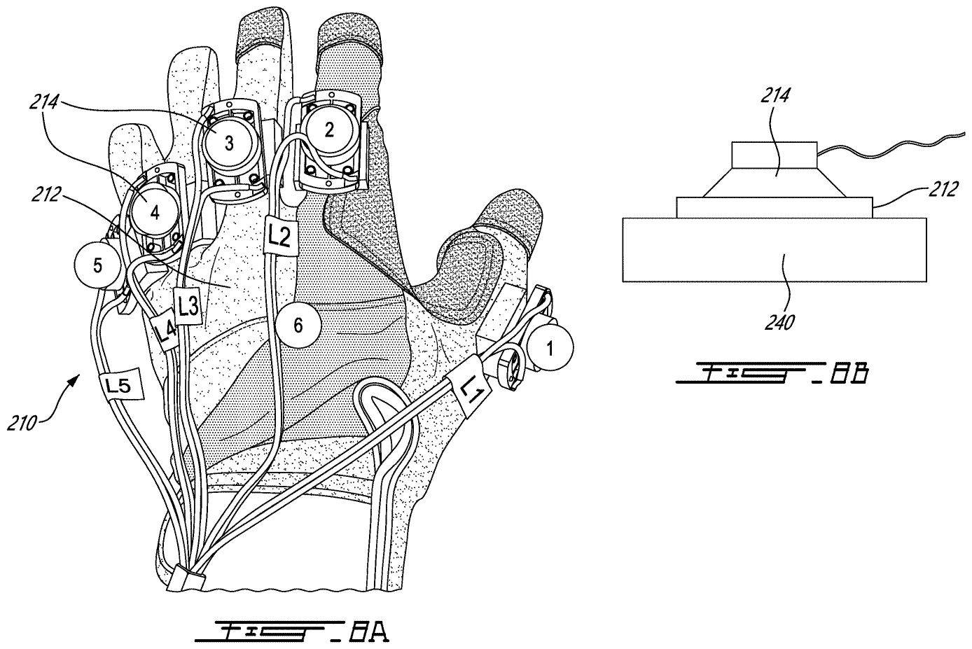

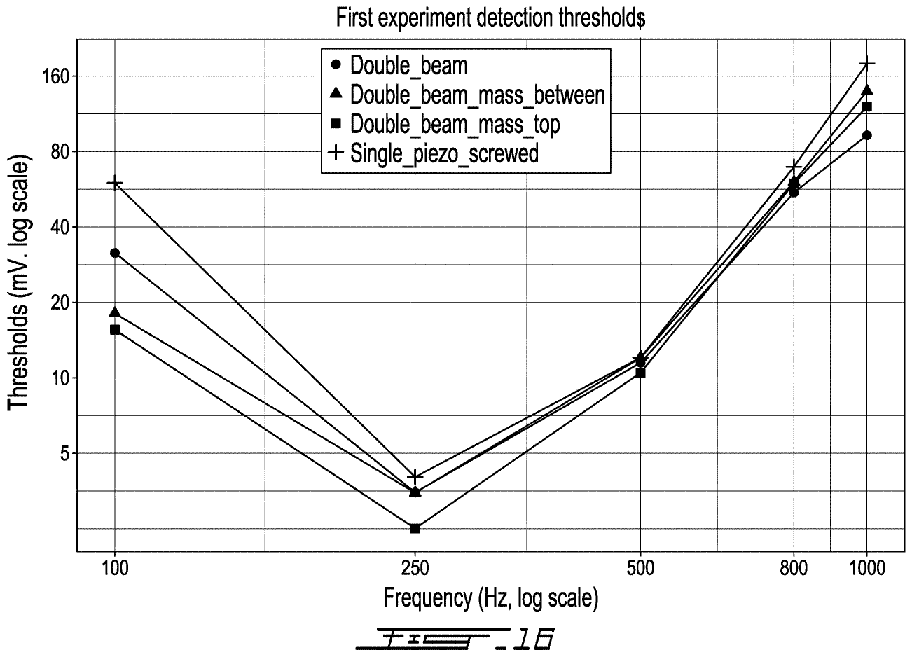

FIG. 1 shows an example of a system 10 for imparting tactile sensations to different tactile regions of a hand of a user. The system 10 can generally be seen to include a garment 12 adapted to be worn by a hand of a user, and a plurality of transducers 14 secured to the garment at different locations thereof. The garment 12 can be in the form of a glove for instance, with or without finger tips, or can be in any other suitable form such as an arrangement of one or more straps or rings. The transducers 14 are configured to generate mechanical displacement based on an electrical signal. The mechanical displacement can generate tactile sensations when the transducers 14 are mechanically coupled to corresponding tactile regions of the user's hand. The garment 12 can be configured to provide the mechanical coupling when worn by the user. For instance, the transducers 14 can be arranged in a manner for vibrations generated by the transducers 14 to be conveyed to the user's skin and felt by the user's skin as tactile sensations. The electrical signal can be conveyed to the different transducers 14 by electrical wires 16, electrical conductors or the like. Some form of electronic device 18 or system can also be provided to generate one or more signal conveyed to the different transducers 14. The different transducers 14 can be driven by the same signal, or by different channel signals having different intensity, frequency and/or temporal acoustic traits. Various examples of a suitable electronic devices or systems will be discussed below. The electronic device 18 or system, or components thereof, can be integrated to the glove or connected to it or to one another in a wired or wireless manner. The transducers 14 can be audio transducers having an electromagnetic assembly including a permanent magnet and a voice coil which moves relative the permanent magnet based on variations in electrical current intensity (an example of which will be presented below in relation with FIG. 8B), or piezoelectric transducers having a body or arrangement of piezoelectric material which mechanically deforms based on changes in electrical potential, for instance (examples of which will be presented below in relation with FIGS. 16, 25, 26 and 27). There are different types of piezoelectric materials, a first type bends in response to changes in electrical potential, and a second type grows or shrinks in response to changes in electrical potential, for instance.

In a first embodiment, such a system 10 can be used to impart different tactile sensations to different tactile regions of a hand. More specifically, a first one of the transducers 14 can be driven via a first channel signal, and a second one of the transducers 14 can be driven via a second channel signal. The first channel signal and the second channel signal can both have one or more of intensity, frequency, sharpness and temporal acoustic traits (e.g. any combination or sub-combination of these four types of acoustic traits). The first channel signal can be distinct and independent from the second channel signal in the sense that its acoustic traits can be different from the acoustic traits of the second channel signal at a given moment in time, and evolve differently and independently. A third channel signal, a fourth channel signal, and even more channel signals having different acoustic traits from all other channel signals can also optionally be provided and used to drive corresponding transducers mechanically coupled to further tactile regions of one or both hands of the user. In some cases, one or more of the channel signals can be used to drive two or more transducers. Examples of different combinations positions for the different transducers on the user's hand or hands will be provided further below.

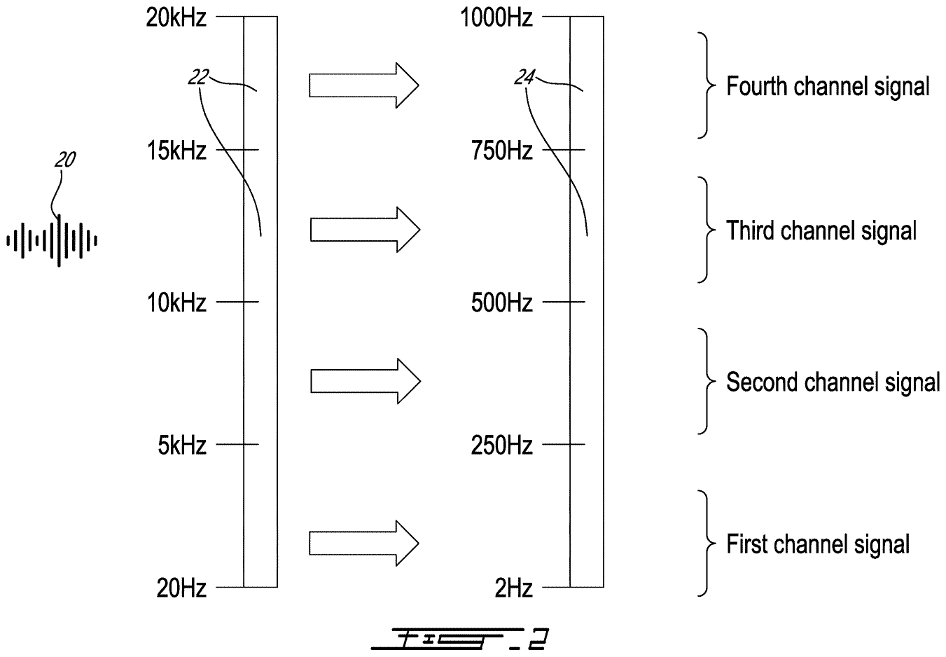

FIG. 2 provides a schematic representation illustrating a first example way of providing different channel signals which can be used to drive different transducers 14 mechanically coupled to different tactile regions of a hand or hands of a user. In this example, a source audio signal 20 (or source audio data) is provided having acoustic traits in an audio frequency band spanning 20 Hz to 20 000 Hz (20 kHz). The frequency band is split equally, or roughly equally, into four partial audio frequency bands 22. In this example, a first of the partial audio frequency bands 22 spans 20 Hz to 5 kHz, a second spans from 5 kHz to 10 kHz, a third spans from 10 KHz to 15 kHz, and a fourth spans from 15 kHz to 20 KHz. Each one of these partial audio frequency bands 22 is then frequency shifted and frequency scaled, as per a transfer function, and mapped against a corresponding partial frequency band 24 in the tactile frequencies between 2 Hz and 1000 Hz. Accordingly, the frequencies in the first partial audio frequency band can be frequency shifted and frequency scaled to corresponding frequencies in a first partial tactile frequency band between 2 Hz and 250 Hz, the second to a second partial tactile frequency band between 250 Hz and 500 Hz, the third to between 500 Hz and 750 Hz and the fourth to between 750 Hz to 1 kHz, for instance, leading to 4 different channel signals.

In the example schematically represented in FIG. 2, it will be noted that the audio frequency band is split equally on a linear scale, and so is the tactile frequency band. However, it is known that the human ear has a sensitivity which scales on a logarithmic scale. Accordingly, in some embodiments, it can be preferred to split the audio frequency band and/or the tactile frequency band, and/or to perform the mapping, in a logarithmic manner, and to provide a corresponding transfer function.

It will also be noted that in practice, only a portion of the “audio frequency band” is actually used for music or speech, such as between 30 Hz and 8 000 Hz, or even between 100 Hz and 4000 or 5000 Hz. In other embodiments, one may want to even further limit the source audio frequency band for reasons associated to a particular embodiment or other preferences. The example above was provided on the basis of the entire audio band simply for illustration of one possibility. It will also be noted that in the example provided above, a distinction between portions of frequency bands is made for the purpose of assisting in the understanding of the underlying concepts. This distinction is tied to the step of splitting different portions of the overall frequency band into different channels. It will be noted that in different embodiments, the frequency bands which are split to different channels, while remaining different, may be partially overlapping. For instance, a first audio frequency band may span 20 KHz to 13 kHz, and a second audio frequency band may span 17 kHz to 8 kHz, to name one possible example. Similarly, there may be discontinuities in the frequency spectrum covered by the transfer function. For instance, in one embodiment, the audio frequency band extending between 14 and 16 kHz may not be mapped to any tactile frequency band or channel (e.g., a first audio frequency band may extend from 20 kHz to 16 kHz whereas a second audio frequency band may extend from 14 kHz to 10 kHz). Moreover, the distinction between distinct frequency bands may appear only at the time of dissociating different frequency bands into corresponding channels, and the transfer function mapping audio frequencies to tactile frequencies may be continuous over the overall frequency spectrums.

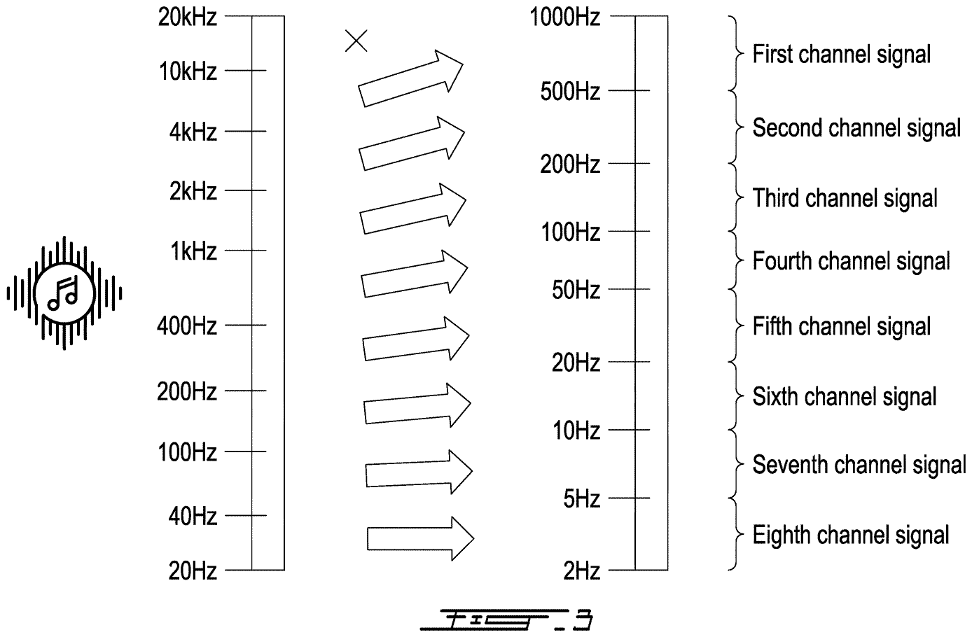

FIG. 3 presents a schematic representation a second example way of providing different channel signals. In this second way, the audio frequency band and the tactile frequency band are mapped to one another on a logarithmic scale rather than on a linear scale, and the corresponding transfer function can correspondingly be logarithmic. The splitting of the tactile frequency bands into corresponding channels is also logarithmic in this embodiment. Moreover, one of the partial audio frequency bands, between 10 kHz and 20 kHz, is ignored from the point of view of the mapping, and is not translated into a channel signal. This illustrates that in some embodiments, only portion of a source signal may be translated into channel signals.

The mapping schemes presented in FIGS. 2 and 3 are tonotopic as opposed to somatopic. Such mapping schemes can be particularly interesting in some embodiments in the sense that they mirror the way the human audition system works, and more specifically to the way the cochlea is mapped to the cortex of the human brain.

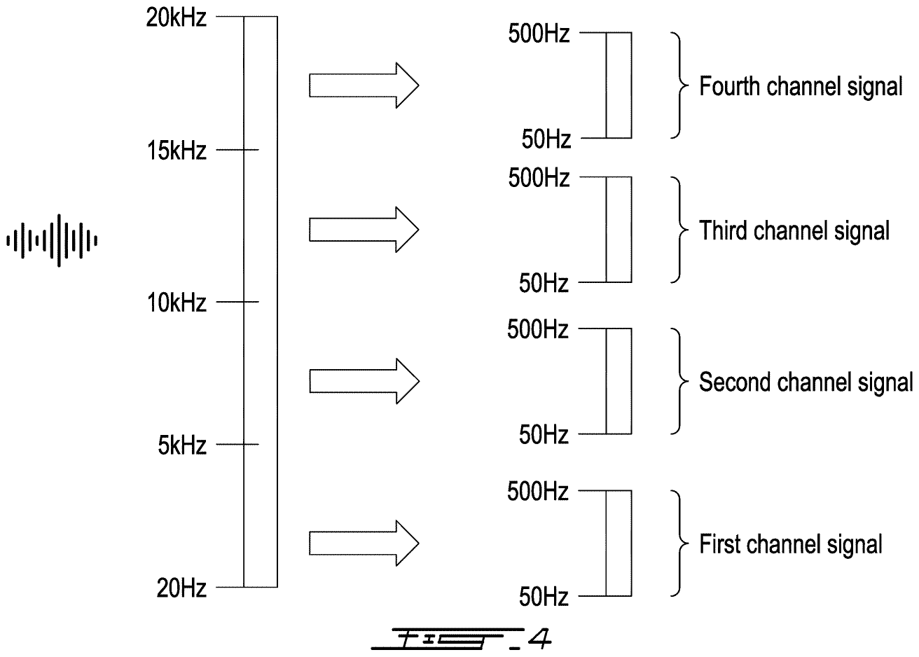

FIG. 4 presents a schematic representation of a third example way of providing different channel signals. In this third way, the source audio signal frequency band is split into partial frequency bands in a manner similar to the first way illustrated at FIG. 2, but the frequency shifting and frequency scaling, or “mapping” to the different channels, is performed differently. More specifically, each one of the different partial audio frequency bands are mapped to a same tactile frequency band associated with a corresponding channel signal. Here, since the acoustic traits in the different partial audio frequency bands are different from one another, the acoustic traits in the different channel signals are also different notwithstanding the fact that they are all mapped within a same frequency band. For instance, a pitch feature at 17 kHz can be mapped to a pitch feature at 200 Hz of the fourth channel signal, whereas a simultaneous pitch feature at 6 kHz in the source signal can be mapped to a pitch feature at 100 Hz of the second channel signal, and these pitch features, which were different in the source audio signal, can remain different in the channel signals. A pitch feature at 16 kHz can be mapped to a pitch feature at 100 Hz in the fourth channel signal, for instance.

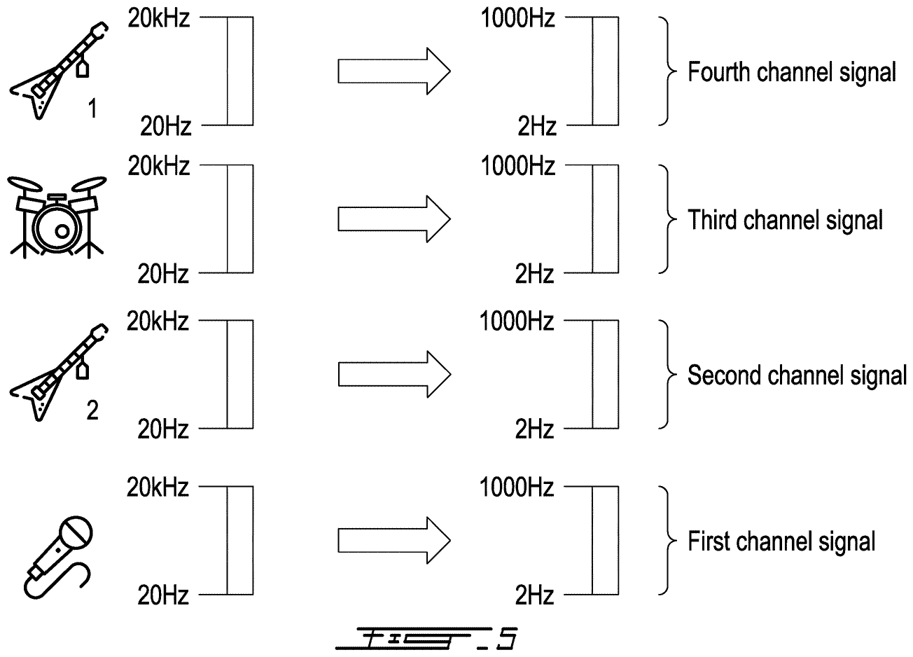

FIG. 5 presents a schematic representation of yet a fourth example way of providing different channel signals. In this example, the source audio signal is split into different tracks based on the instruments which collaborate to generate music. Each instrument is associated a given track, or source audio signal, which can cover the entire audio frequency band (e.g. 20 Hz-20 kHz) or only a portion of the audio frequency band. The partial signal associated to each track is frequency shifted and frequency scaled to a tactile frequency band and can be outputted as a distinct channel.

It will be noted that frequency shifting can refer to the process of shifting each frequency to another frequency, by a given frequency value (e.g. each frequency is transformed to =base frequency+x frequency value where x represents the frequency shift), whereas frequency scaling refers to the process of scaling a first frequency band having a first frequency span (e.g. a frequency band extending from 15 kHz to 20 kHz spans 5000 kHz) to a second frequency band having a second frequency span (e.g. a frequency band extending from 2 Hz to 1000 Hz and spanning roughly 1000 Hz). In a scaling process which leads from a larger frequency span to a smaller frequency span, the frequencies within the initial band are compressed in the transformation, whereas in a scaling process which leads from a smaller frequency span to a larger frequency span, the frequencies within the initial band are expanded/spaced apart from one another in the transformation. Either one, any subcombination, or all of the frequency splitting, frequency scaling, and frequency shifting functions can be performed on a linear basis or on a logarithmic basis, to name two examples.

The above-illustrated functions mapping audio frequencies to tactile frequencies, such as frequency shifting, and/or frequency scaling, and splitting into different channels, or frequency splitting, can be collectively referred to as frequency-based processing, or just “processing” for short, or more specifically referred to as a step of applying a transfer function. They can be performed in an automated manner using electronic equipment such as a computer. The hardware and/or software elements responsible for performing the processing function can be referred to as a processing module or transfer module for simplicity. Similarly, the function of driving the transducers with the channel signals can be performed in an automated manner using electronic equipment such as a computer or other electronics. The hardware and/or software elements responsible for performing the driving function can be referred to as a driving module for simplicity.

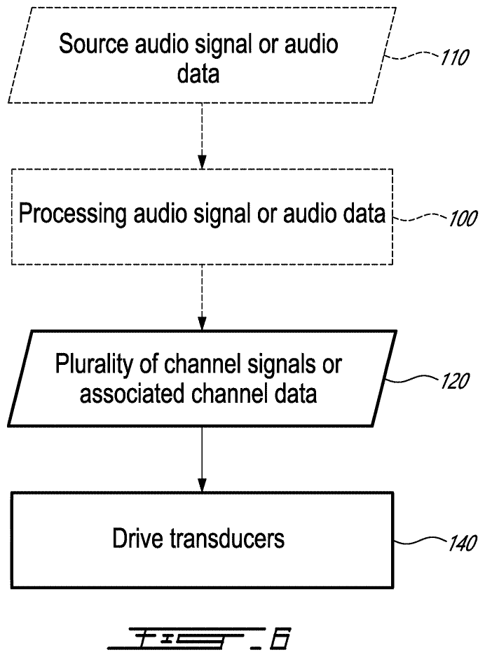

Referring to FIG. 6, in many embodiments, a step 100 of processing a source audio signal or source audio data 110 will be used to transform a source audio signal or source audio data 110 into the plurality of channel signals or channel data 120 associated thereto, which are used in the process of driving the transducers 140. However, the step of processing a source audio signal may or may not be performed by the same electronic equipment than the electronic component(s) responsible for driving the transducers. Indeed, the step of processing the source audio signal or associated audio data 100 may, in some cases, be performed at a significantly different moment in time than the step of driving the transducers, such as at a different hour, a different day, or even a different month or different year. Moreover, in some embodiments, the step of processing the source audio signal may even be entirely omitted, as some embodiments may be suited to using channel signals, or associated channel data, which was prepared independently of any source audio signal or associated audio data. The expression “signals” refers to electrical or electromagnetic fluctuations in a media (such as electromagnetic waves propagating in air or electrical current circulating in a conductor) which occur in real time, whereas the expression “associated data” refers to data which is stored in a computer memory, such as a persistent computer memory, and which can be executed upon by a processor or other electronic equipment to generate a signal.

Returning to FIG. 1, the driving module 26 which is used to perform the function of driving the transducers 14 based on the channel signals, can either be adapted to receive one or more associated signals, and potentially amplify them, and output them to the transducers 14, or can be adapted to generate signals based on data stored in a computer memory 28 for instance. The driving module 26 can consist of one or more component modules for instance. For example, the driving module 26 can have a plurality of component modules configured to amplify and/or output corresponding ones of the channel signals for instance. The optional processing module 30 can either be adapted to receive one (or more) source audio signal 32 or to read associated data stored in a memory 28. The processing module 30 can be adapted to output a plurality of channel signals, or to output associated data stored in a memory which can, in one way or another, eventually be made available to the transducers 14 via the driving module 26.

Humans interact with the world through their senses. Auditory perception is the primary gateway for communicating with others through language and music. However, the human environment being rarely unisensory, other senses can support or alter this perception. Multisensory interactions between auditory and tactile are likely to exist given that these two modalities are responsive to similar physical stimulations, namely, mechanical pressure in the form of oscillations such as acoustic waves. Bearing in mind that some activities that produce tactile sensations also produce sound, a system for imparting tactile sensations to a hand of a user can be provided in the form of a garment 12. In one example, the garment 12 can be in the form of a glove containing vibrotactile loudspeakers with a plurality, e.g., ten or twelve to name two possible example, of independent channels to study auditory-tactile interaction in humans.

A mono glove (1 glove, 1 channel) can allow the exploration of musical perception in individuals with extensive multisensory training (professional musicians) and/or long-term auditory deprivation. Experiments made on this basis revealed that all participants (controls, professional musicians and individuals with long-term auditory deprivation) were able to discriminate frequency and identify emotion in music via tactile stimulation only. Furthermore, the results suggest that individuals with extensive musical training have improved performance for auditory-tactile and tactile frequency discrimination in comparison to a control group. Additionally, individuals with profound long-term auditory deprivation were better at identifying happiness in music with tactile stimulation (in comparison to normal-hearing participants) suggesting compensatory plasticity in another modality. A more complex system may offer new functionalities and insights. For instance, a system using two vibrotactile gloves with multiple transducers that can transmit different and distinct sound information to different tactile regions such as different fingers. Such a more complex system can allow for the creation of different patterns of frequency representation of sounds on the hands. For example, low, medium, and high-pitched sounds could be found on different fingers to reproduce the tonotopic organization of the cochlea and, for example, facilitate the discrimination of different frequencies in music. The stimulation of both hands could offer redundancy and/or segregation of sensory information depending on the context.

It may also be promising to investigate tactile perception following long-term musical training even if haptic cues are known to be an essential part of musical production. Indeed, vibrations felt at the fingertips of professional musicians while playing their instrument (piano or violin) may lead to an increase in perceived sound loudness and richness. Extensive musical training can improve auditory-tactile and tactile frequency discrimination. Regarding musical timbre, using a haptic chair, non-musicians with and without hearing impairment can discriminate musical instruments via the tactile modality alone. However, timbre perception has never been studied behaviorally from a multisensory standpoint including both auditory and tactile modalities. Furthermore, it may be promising to explore neural activity elicited by auditory-tactile and tactile perception of this important musical parameter. In particular, one may want to determine whether the improvement of performance measured following multisensory training (MST) results from better multisensory integration (optimized communication between the senses) or from the addition of improved unisensory performance, for instance. To better understand the impact of multisensory training on auditory scene analysis, as well as to understand how the different sensory systems communicate, a multimodal and multi-technique approach may be required, such as the one described herein.

Moreover, a system 10 for imparting tactile sensations to different tactile regions of a hand of a user can offer many possibilities for applications. People with hearing disorders may benefit from this technology to improve their sound perception, especially in noisy environments. Musicians may also use this technology to better understand and interpret music by feeling the different frequencies on their fingers. Overall, vibrotactile gloves offer a new path to improve sensory perception, opening the door to many potential applications in various fields such as medicine, music, and assistive technology.

The tactile system of the human body is typically able to sense vibrations with frequencies ranging from below 20 Hz, such as about 0.4 Hz, above 500 Hz, such as up to 1000 Hz. The most sensitive frequency range lies between 40 and 1000 Hz, more specifically between 200 and 300 Hz. Pacinian corpuscles are the primary neural receptors involved in vibration perception. Pacinian corpuscles (FA-II) are Rapidly Adapting type Low-Threshold Mechanoreceptors that are highly sensitive to vibrations in the range of 100 to 1000 Hz. For these corpuscles, the known highest frequency sensitivity point is 250 Hz (threshold is lower at this frequency) and significantly increased with each lowering and increasing frequencies. For both Pacinian corpuscle afferents in glabrous and in hairy skin, the measured thresholds produce a U-shape as a function of frequency. For vibrotactile applications, these corpuscles may need to be particularly targeted to transmit sound through touch.

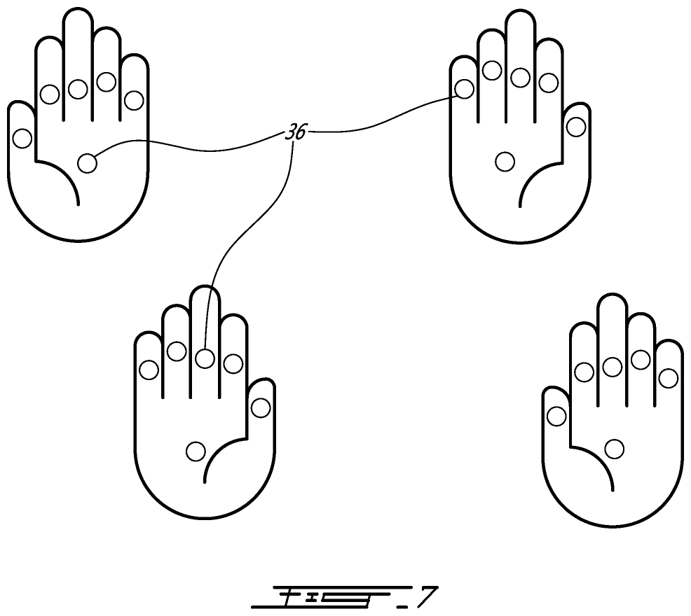

Turning now to FIG. 7, a plurality of circles 36 are shown on palmar faces and dorsal faces of hands of a user. Such circles illustrate a set of example tactile regions on hands of a user to which corresponding transducers may or may not be mechanically coupled. Other tactile regions of the hands of a user exist to which transducers may be mechanically coupled, and the ones illustrated in FIG. 7 are provided only for the purpose or exemplification. It will be understood based on the above that different tactile sensations can be imparted to different regions of one or more hand of a user by driving transducers mechanically coupled to the respective regions by respective channel signals, and where the different channel signals have different acoustic traits such as differences in intensity, differences in frequency, temporal differences, any sub-combination thereof, or a combination thereof.

In one embodiment, for instance, one may wish to apply a fundamental frequency Fo to a tactile region located on the palm of a user, and to distribute different portions of the audio frequency spectrum to different ones of the fingers such as thumb 100 Hz-250 Hz, index 250-400 Hz, middle finger 400-650 Hz, ring finger 650-800 Hz, and little finger 800 Hz-1000 Hz in a combination of a tonotopic and somatotopic organisation which may be useful in some embodiments. Optionally, harmonics or a fundamental frequency can be modified in the sounds presented to the different hands, in an independent manner from one transducer to another.

FIGS. 8A and 8B present an example embodiment of a system 210 for imparting tactile sensations to different tactile regions of a hand 240 of a user. In this example embodiment, the system includes a garment 212, namely a glove, configured to be worn by a hand 240 of a user, and a plurality of actuators provided here in the form of electromagnetic audio transducers 214 (speakers). The audio transducers 214 are secured to corresponding locations of the garment 212. In this embodiment, two gloves are used (only one is shown, the other being a mirror image of the first), and corresponding audio transducers 214 are mechanically coupled to the backsides of each one of the fingers (including the thumb), and to the palm region. Each one of the audio transducers 214 has a permanent magnet, a voicecoil, and a diaphragm extending between the voicecoil and the permanent magnet. Based on changes in electrical current circulation around the voicecoil, an active magnetic field is generated which interacts with the passive magnetic field of the permanent magnet and moves the voicecoil, and the diaphragm, along an axis, producing sound. In this embodiment, the sound is conveyed in the intervening media of air and glove material and reaches the user's skin at the tactile region.

Experiments were conducted using a system 210 such as presented in FIGS. 8A and 8B, the results of which are presented in FIG. 8C. The as can be seen from the figure, actuators that were positioned on the dorsal side of the different fingers, including the thumb, did not produce significantly different thresholds between each other at any frequency. This provides an indication that a homogeneous stimulation can be provided to all fingers. Significantly higher thresholds were consistently measured for the palm at the lowest (100 Hz) and highest frequency conditions (1000 Hz), where it performed significantly worse than either one of the Index and Middle fingers, even though in theory, the palm is made of highly sensitive glabrous skin and could be expected to be more sensitive. A physiological hypothesis is that the underlying tissue may play a more important role, as vibrations are better transmitted over bone than soft tissue, particularly at higher frequencies. This would mean that transducers can efficiently transmit vibrations to the population of Pacinian corpuscles concentrated in the connective tissues near the joints and bones of the proximal phalange. It should be noted that actuators on the palm also limit the user's ability to rest their hand on a surface, which could be inconvenient, especially for extended use.

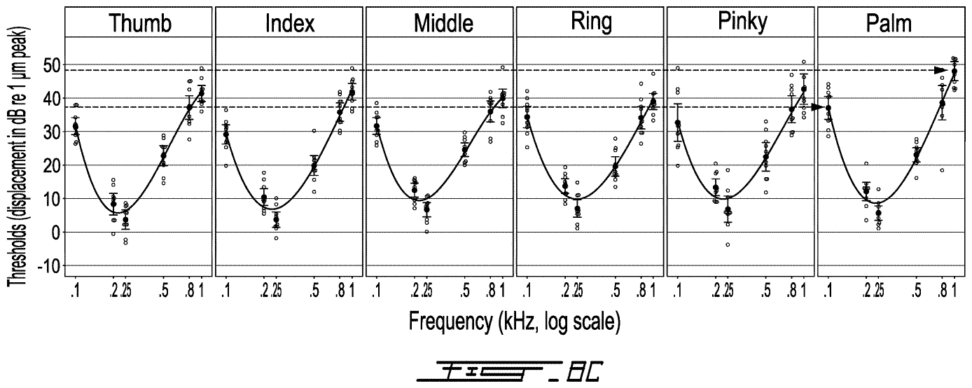

A system such as presented in FIGS. 8A and 8B can be represented by a lumped-parameters model with single degree of freedom (1 DoF), as schematically depicted in FIG. 9. Such a model can be used to analyze the vibration behaviour of the system. In this model, k and c represent the stiffness and damping effects of the loudspeaker and the glove together. The system is excited by the audio signal y(t) applied to the loudspeaker. The proposed experimental setup is capable of measuring the acceleration of the loudspeaker y and the force transmitted to the finger F_{T}. According to the model, the differential equation of motion can be obtained by:

$$m \ddot{x} + c ( \dot{x} - y ) + k ( x - y ) = 0 \qquad ( 1 )$$

The force transmitted to the finger (F_{T}) can be calculated by

$${f}_{t} = - c ( \dot{x} - \dot{y} ) - k ( x - y ) \qquad ( 2 )$$

By modelizing the vibrations as harmonic, the variables are defined by

$$\begin{matrix}x ( t ) & = {Xe}^{i ω t} \\ y ( t ) & = {Ye}^{i ω t} \\ {f}_{T} ( t ) & = {F}_{T} {e}^{i ω t}\end{matrix} \qquad ( 3 )$$

By replacing the exponential terms in the differential equation of the motion, one can obtain:

$$\frac{{F}_{T}}{k Y} = {r}^{2} T R \qquad ( 4 )$$

where

$$r = \frac{ω}{{ω}_{n}}$$

is the amplitude ratio and

$$ζ = \frac{c}{2 m {ω}_{n}}$$

is the damping ratio of the entire system. TR represents the transmissibility of the displacements and can be obtained by:

$$TR = \frac{X}{Y} = \frac{\sqrt{1 + {( 2 ζ r )}^{2}}}{\sqrt{{( 1 - {r}^{2} )}^{2} + {( 2 ζ r )}^{2}}} \qquad ( 5 )$$

Moreover, by applying an accelerometer on the speaker, one can experimentally measure the behaviour of the system.

Two criteria may be used to detect a resonance peak for the system:
1. There is a peak in the amplitude;
2. There is a remarkable phase angle variation around the peak.

Based on these criteria and on the experimental results, a resonance frequency of the system is obtained around fₙ=3200 Hz. Table 1 summarizes the experimental results:

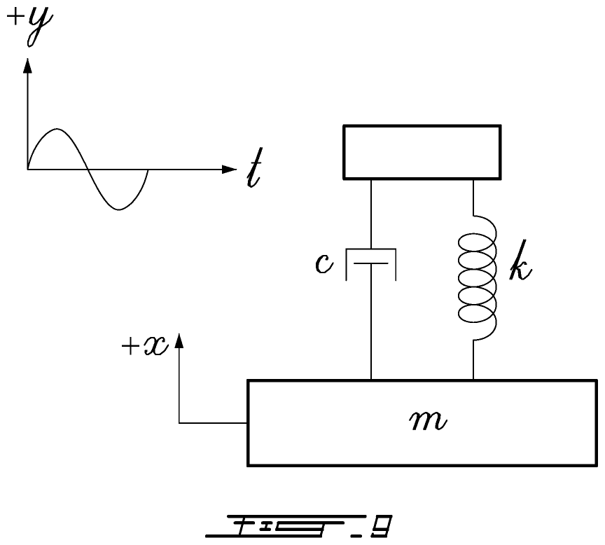

TABLE 1
summary of experimental results of FIG. 10

| | Measured |
|---|---|
| Parameters | values |
| Resonance | 3200 Hz |
| Frequency | |
| Maximum | 0.0108N |
| Force | |
| Maximum | 0.3834 g |
| Acceleration | |

Based on the above, it will be noted that in some embodiments, traditional, electromagnetic, audio transducers may be suitably used for imparting tactile sensations to different tactile regions of a hand of a user. In particular, in medical or scientific applications, it may be relevant to map the brain response to tactile stimuli. Such mapping of the brain response can be acquired using magnetic resonance imaging (MRI). For instance, one may want to impart tactile sensations to different tactile regions of a hand of a user while a head of the user is undergoing MRI, such as illustrated in FIG. 10.

Considerations may be made to ensure MRI compatibility of a system for imparting the tactile sensations. Indeed, when undergoing an MRI, any sport clothing or any clothing labeled as antimicrobial, antibacterial, or as containing metallic particles (e.g., silver) should be avoided. These fibers can trap heat and sweat, which can eventually lead to thermal burns on the skin. More than 70% of MRI complications are related to thermal burns. The most effective way to avoid burns injuries when undergoing an MRI is to use natural fibers, such as cotton or linen. Other common materials that are safe for MRI include glass, nylon, Teflon, and various plastics. Generally, it can be preferred to avoid using a system which would impart significant electromagnetic interference (EMI) into the function of the MRI.

In particular, ferromagnetic materials, such as the permanent magnet which is typically used in traditional audio transducer design, are contraindicated. This leaves a want for a system which would offer MRI compatibility.

A portion of the challenge can be addressed by an approach such as schematized in FIG. 1, where all the electronic components with the exception of the transducers themselves may be provided remotely rather than incorporated to the glove itself (though incorporating to the glove itself may remain a valid approach in some applications), and can be connected to the glove by wires for instance.

Another portion of the challenge can be addressed by using suitable materials for the garment which is configured to be worn by the hand of the user, or for securing the transducers to the garment. Indeed, the garment can be provided in natural fibers rather than synthetic fibers, or specifically be made of nylon. In particular, it may be desired to use nylon-based hook-and-loop fasteners such as Velcro™ to secure the garment or to apply a pressure to force or trap the transducer into closer proximity or direct or indirect contact with the tactile region of the user's hand. Similarly, cyanoacrylate glue is considered suitable for use in a MRI context and can be used to secure different components of the garment to one another, and/or to secure the transducers to the garment.

However, these solutions alone do not address the issue of the presence of the permanent magnet as part of the audio transducer itself. This being said, it was found that the latter issue can also be addressed, more specifically by using a piezoelectric material as the transducer (e.g. a piezoelectric transducer) as opposed to a permanent magnet, voice coil and diaphragm assembly.

To test this hypothesis, an experiment was conducted where four piezoelectric transducers were attached to a garment. Table 2 lists the characteristics of the actuators.

TABLE 2
Characteristics of the piezoelectric actuators for a load with a mass of 100 g

| | | Dimensions | Voltage | Mass | Displacement | Force | Capacitance |
|---|---|---|---|---|---|---|---|
| No. | Type | [mm³] | [V] | [g] | [μm] | [N] | [μF] |
| 1 | Bimitech Piezo | 50 × 20 × | 160 | 1.58 | 420 | 0.49 | 0.071 |
| | Bender | 0.54 | | | | | |
| | PUA3020-5H200 | | | | | | |
| 2 | TDK Mini | 12 × 4 × 1.8 | 60 | | 27 | 0.10 | 0.42 |
| | PowerHap | | | | | | |
| | 1204H018V060 | | | | | | |
| 3 | TDK PowerHap | 9.0 × 9.0 × | 60 | | 35 | 0.20 | 0.85 |
| | 2.5G | 1.1 | | | | | |
| | 0909H011V060 | | | | | | |
| 4 | TDK Mini | 9 × 3.75 × | 60 | | 12 | 0.25 | 0.35 |
| | PowerHap | 1.4 | | | | | |
| | 0904H014V060 | | | | | | |

The experimental setup was similar to the one presented above with audio transducers.

Accelerometer readouts of tests using this setup were obtained in the time domain and in the frequency domain. According to the time domain, the maximum vibration acceleration is about 2.2 g in which g is the gravitational acceleration. In addition, the maximum transmitted force is about 0.005 N which is much lower than the force transmitted by the loudspeaker setup. According to the frequency domain, the maximum transmitted force is at the frequency of 900 Hz. Although the acceleration level is fairly high over a wide range of frequencies, the transmitted force is only significant for a relatively narrow bandwidth around the peak. This arrangement alone may not be suitable for delivering enough energy to impart sufficient forces at higher frequencies.

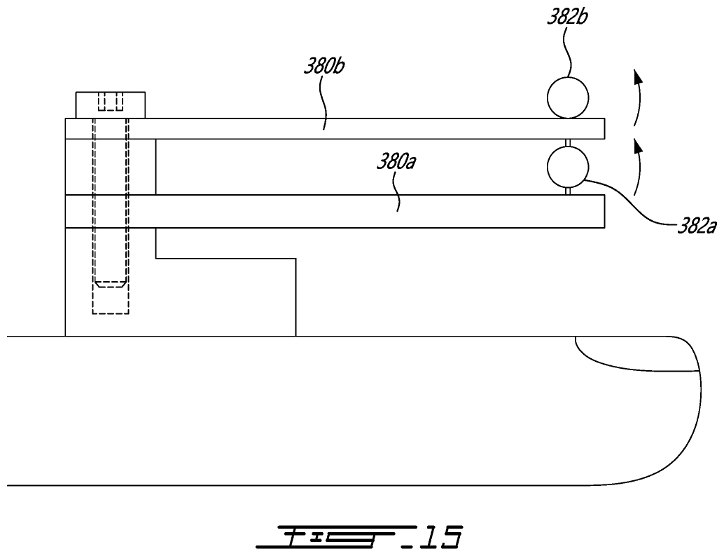

According to the experimental modal analysis results presented at FIG. 15 for piezoelectric transducer no. 1, the first natural frequency peak of the piezoelectric system is located around the frequency of 2600 Hz. However, at this frequency the coherence is relatively weak (around 50%) which shows that this peak might not be reliable as the level of the measured noise is high. The second peak is located around the frequency of 4900 Hz for which the associated coherence is quite strong and hence the peak is more reliable.

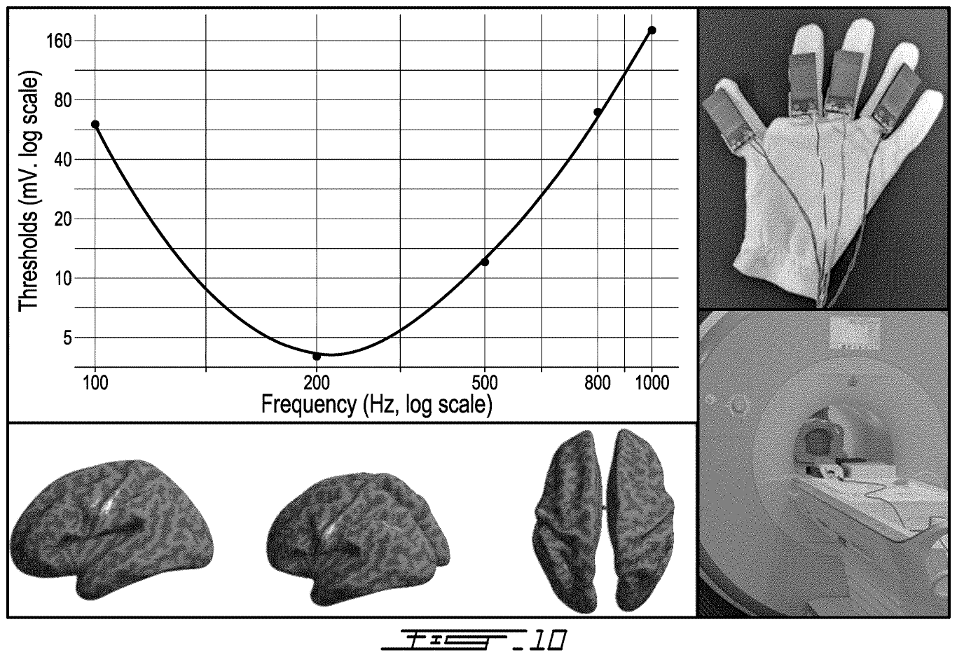

A first approach to mechanically coupling a piezoelectric transducer 314 to a tactile region is presented in FIG. 11. In this case, a piezoelectric element is integrated into the garment 320 tissue or sandwiched against the tactile region by the garment 320. The lumped-parameter vibration model of the piezoelectric vibrotactile glove can have each part modeled by a single-degree-of-freedom vibration module consisting of a mass, spring and damper designated by m, k and c.

This lumped-parameter model contains 3 degrees of freedom (DOF). The force F_P applied by the piezoelectric element driven by the audio signals is transmitted to the finger. The transmitted force F_T can be analyzed to be large enough to be sensed by the user.

This 3-DOF model can be simplified to a single DOF model having fewer elements and being less complicated for the analysis, but can still represent the main features of the real system. The simplified model takes into account the inertia effect of all the masses in the 3-DOF model by one single block whose mass is m_{eq}. The stiffness and damping effects of the glove and the piezoelectric element are still conserved in the simplified model, however those related to the finger can be ignored or incorporated into the other spring or damping elements of the simplified model.

The simplified model is then investigated to find the optimal values of k_{G} that makes the amplitude of the transmitted force F_{T} sufficiently large over a wider range of frequencies. The proposed simplified model was numerically simulated in the Simulink toolbox of Matlab. According to the results, the higher is the garment's stiffness, the wider is the range of frequencies at which a minimum sensible force (let's say 0.1 N) can be transmitted to the finger. It means that there is no optimal value for k_{G} since the higher value of k_{G} is more desirable.

This conclusion would be correct if the piezoelectric element could deliver the same amplitude of generated forces F_{P} regardless of the stiffness of the garment 320. However, it is not the case in reality. The amplitude of the generated piezoelectric force become more limited as the garment tension force becomes more significant.

To include this effect to the model, an electrical model for the piezoelectric element can be developed. In this model the resistor R is part of the amplifier used to drive the device. The nonlinear capacitance Cₕ is required to model the hysteresis phenomenon of the piezoelectric device. C_{P} represents the linear capacitance of the piezoelectric element and Tₑₘ is the transducer constant. As the current q flows into the circuit, charge develops across the working capacitor C_{P} which produces the force F_{P} due to the inverse piezoelectric effect. Conversely, any resulting displacement x in the piezoelectric transducer generates a current \dot{q}_{P} in the circuit due to the piezoelectric effect.

The nonlinear model can be simplified by ignoring the piezoelectric hysteresis behaviour and removing the nonlinear capacitance element. It makes the entire system linear while maintaining the most important electrical elements in the model.

Finally the simplified 1-DOF vibration model and the simplified linear electrical model can be combined to build a comprehensive electromechanical model of the piezoelectric vibrotactile glove.

This model can be investigated to find the optimum value of kc equivalent to the glove tension force to ensure that the transmitted force can be sensed over a wide range of frequencies. The mechanical characteristics of the piezoelectric device can be obtained through the catalogue of the piezoelectric actuator manufacturer or by using the modal analysis results studied in the previous section. The electrical parameters of the system can be measured by conducting more tests on the piezoelectric elements.

The simplified electromechanical model of the piezoelectric vibrotactile glove was simulated in Simulink. The mechanical and electrical subsystems are connected through Tₑₘ which is the transducer constant of the piezoelectric actuator.

To validate the electromechanical piezoelectric model, the Piezo Haptic Actuator with PZT ceramic, Type 1204H018V060 is used. The parameters are listed in Table 2 above.

The developed piezoelectric simulink model is then simulated with a half wave sinus signal form of amplitude 0 . . . 60V and pulse length of 5 ms which is equivalent to 200 Hz. The acceleration results as a function of input voltage with 100 g load. According to simulation and experiment, there is a perfect match between the simulation and experimental results which explicitly demonstrates the efficiency and accuracy of the simulation model.

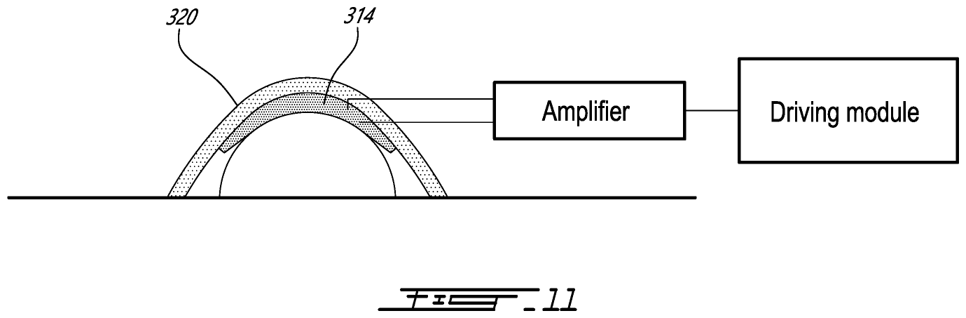

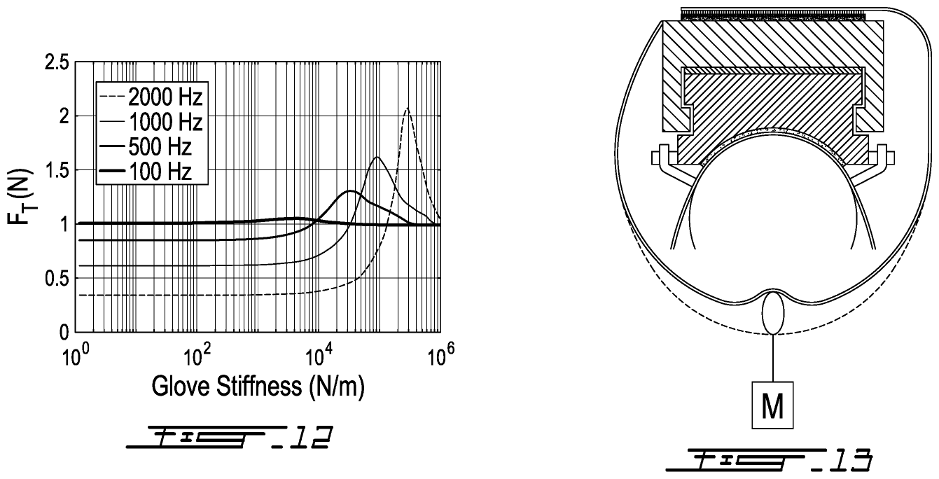

FIG. 12 presents a graph of modelization of the glove stiffness (tightness) on the frequency response of the system. As shown in this graph, higher glove stiffness appear better, but this should be balanced with considerations such as user comfort.

A first proposed design to favour mechanical coupling is presented in FIG. 25.

The proposed design was tested for EMI interference in MRI and showed no susceptibility to magnet gradient, nor did produce any image artefact.

The proposed design was tested by the researchers on one finger and the elastic band tension suggested by the graph presented in FIG. 12 was judged to be uncomfortable.

To address this issue, mass was added on the top of the piezo electric element, to maximize the mechanical coupling through inertance. While this improved the coupling at low-frequency for a reduced band tension, it led to a degradation of performance at high-frequencies (between 800 and 1000 Hz) which may make the approach less suitable for some applications.

Various further configurations and assemblies of the piezo elements (a flat ceramic of 12 mm×30 mm) where conducted to ascertain the potential of combining piezo elements to form a single transducer:

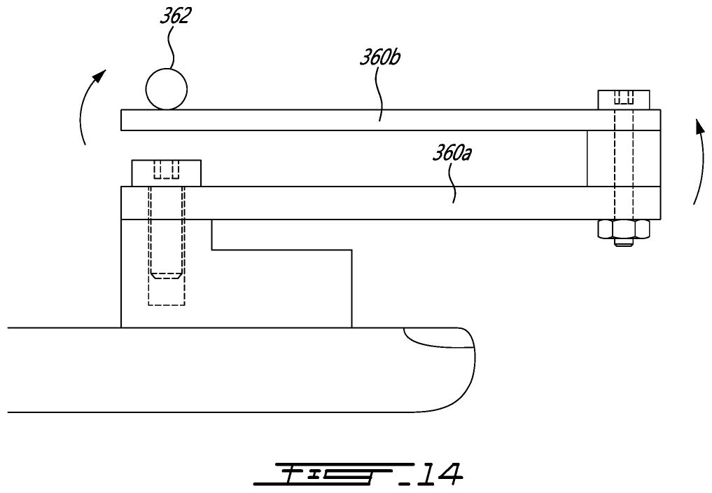

FIG. 14 schematizes a first example where two piezo elements 360a, 360b were assembled in a counter position so that a positive voltage on the piezo elements doubles the magnitude of the flexion and a negative voltage leads to the two piezo element hitting each other. An additional mass 362 may optionally be added.

FIG. 15 schematizes a second example where two piezo elements 380a, 380b were assembled in a parallel position with a residual space in-between. In a first configuration, these 2 elements are powered with the same voltage, and have the same displacement, simply doubling the moving mass, hence increasing the mechanical stimulation. In a second configuration, a mass 382a is pinched between the two piezo stacks, adding intertie to the system. In a third configuration, a mass 382b is added on only one of the two piezo element: such piezo will show a better low-frequency response, while the second piezo exhibits the usual weaker response at low frequency and good response at high frequency.

To asses the psychophysical properties of the system, we evaluated vibration detection thresholds on the human hairy skin. Detection thresholds were measured in two subjects (1 male and 1 female). The subjects wore the garment on the left hand, and the actuator was positioned on the dorsal aspect of the index finger's proximal phalanx. The stimuli consisted of sinusoidal waveforms for 5 frequency conditions covering most of the tactile perceptual range (100, 250, 500, 800, and 1000 Hz). The sinusoid were generated by an analog audio signal generator (NTi Audio Minirator MR-PRO). To determine the optimal design for maximizing tactile sensitivity, we compared the detection thresholds across multiple configurations of the piezoelectric components. In a first series of experiments, 4 configurations were tested with a single piezoelectric element secured on the fixed base plate using adhesive tape:
Single piezo (fixed with tape);
Single piezo and elastic band;
Single piezo and added mass;
Two piezo, one directly on top of the other.

In a second series of experiments, 4 other configurations were tested to evaluate the alternative setup with two piezoelectric elements forming a double beam. In these experiment, the piezoelectric element were secured using two screws (FIG. 15):
Double beam
Double beam and added mass on top
Double beam and added mass between
Single piezo (fixed with screws)

During the experiments, it was observed that the piezoelectric actuator could produce sound, particularly for the higher frequencies tested (e.g., 800 and 1000 Hz). To mask any possible contribution of the auditory system, the subjects wore sound attenuating earphones playing white noise. Since vibrotactile stimuli are known to be prone to habituation effect, the thresholds were established with an ascending series using the method of limit. Conveniently, this method also controls for perseverance effects, which can cause underestimations of the true threshold. The stimuli were presented for around 1 second, if no response was given by the subject, the stimuli would be increased in intensity and presented again for 1 second. This was repeated until the subject reported a sensation, at which point the stimuli would be decreased to a below threshold value and a new ascending series would start. The threshold was established as the average from 3 to 5 ascending trial. Typically, the detection of lower frequencies (e.g., 100 or 250 Hz) was very consistent, necessitating only few trial to converge to a reliable threshold. In contrast, the detection of higher frequencies (800 or 100 Hz) tended to vary between ascending series, often necessitating more trial to establish a reliable threshold.

The result of the threshold measurements for the first experiments are presented in FIG. 16. The shape of the threshold curve matches the expected “U” shaped of vibrotactile thresholds with the lowest threshold around 250 Hz and with elevated thresholds beyond 800 Hz.

All actuator configurations performed similarly well in the 250 Hz and 500 Hz conditions. For the lowest frequency condition of 100 Hz, the configurations that increased the weight of the system (i.e., added mass, double piezo) performed better than the baseline single piezo configuration, while the configuration that increased the rigidity of the system (i.e., elastic band) performed significantly worse. Conversely, the elastic band configuration was among the best in the higher frequency conditions (i.e., 800 and 1000 Hz), whereas the weighted configuration performed the worst. We also observed a significant difference between the two subjects for in the standard single piezo configuration for the higher frequencies. This could be attributed to the glove forming a tighter fit on the subject with larger hands. Interestingly, the double piezo configuration performed equally well in the higher frequency, where it performed similarly to elastic band condition, and in the lower frequencies, where it performed similarly to the additional weight configuration. This led us to perform a second series of experiment with double piezo configurations. For the second series of experiment, a more reliable way to secure the piezoelectric element on the base was also tested.

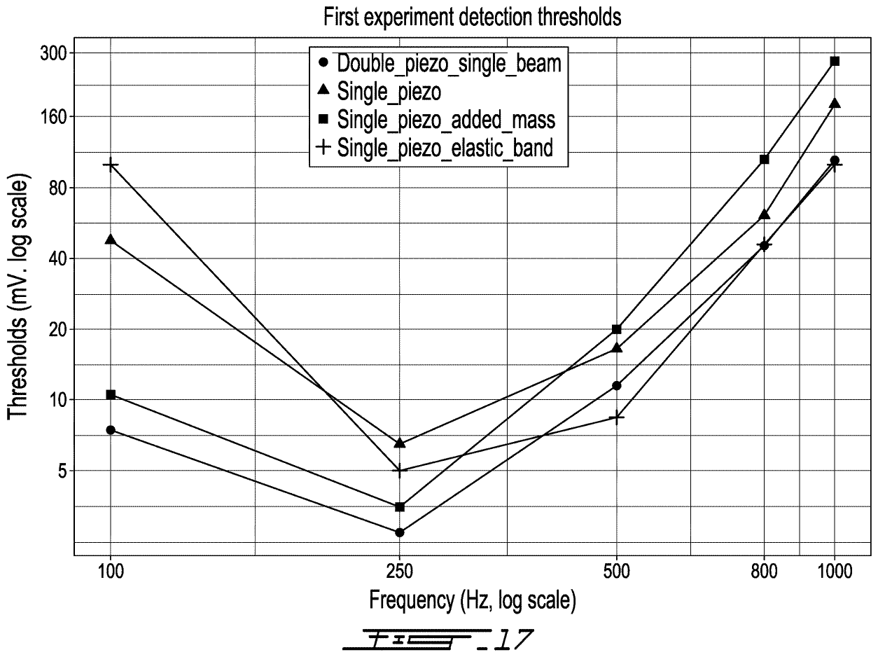

The result of the threshold measurements for the first experiments are presented in FIG. 17. All the configurations in the second series of experiments performed similarly in the 500 Hz and 800 Hz condition. Unsurprisingly, the two configurations with added mass performed better in the lower frequency conditions (i.e., 100 Hz and 250 Hz). The single piezo fixed with screw was generally worse than all other conditions. The simple double beam setup was the best at higher frequencies (i.e., 800 Hz and 1000 Hz) and was average everywhere else. This simple double beam setup seems to strike a good compromise for our requirement of an optimal performance at higher frequencies with an adequate performance at lower frequencies.

Accordingly, in some cases, simply using a piezoelectric transducer alone e.g. with a portion, or its entirety, sandwiched against or otherwise mechanically coupled to the tactile region of the skin, may be deemed to allow satisfactory force feedback, particularly if the imparted vibrations are in the frequency ranges of highest sensitivity of the tactile region, for instance. The spring constant of the “spring” (e.g. garment) which mechanically couples the piezoelectric transducer to the tactile region of the user's hand can be of between 85 and 350 N/m, for instance, and in some cases more specifically between 120 and 250 N/m for instance, 170 N/m being an example of a typical value. The higher the spring constant, the stiffer the coupling and hence the better the force transmission, but on the other hand, this can lead to user discomfort and a higher resonance frequency of the coupled system, which may lead to a want to achieve a balance between the different considerations.

In others, such as in some frequency bands or with certain types or strength of piezoelectric transducers, it may be more difficult to achieve sufficient force transfer to be satisfactorily felt by the tactile receptors of the skin. In such cases, different strategies may be used, such as tightening the transducer against the skin, or using a weight on the transducer. In some cases, using the weight will affect the force in some of the frequency bandwidths and therefore one may wish to use more than one piezoelectric transducer, which each may be individually optimized for a given portion of the overall bandwidth. In some embodiments, a weight having between 1 g and 3.5 g may be found suitable, and more specifically, in some embodiments, a weight having between 2 and 3 g may be found suitable.

It will also be noted that the experiments reported above were performed with a first type of piezoelectric transducer which has a flat sheet which “bends” upon changes in electrical potential. Other types of piezoelectric transducers exist and may exhibit preferable behaviors in the context of a system for imparting tactile sensations to a tactile region of a hand of a user. In particular, another type of piezoelectric transducer “swells” and “shrinks” upon changes of electric potential rather than “bending”. This “swelling” type of piezoelectric transducer may be more expensive than a piezoelectric transducer of the “bending” type at the time of filing this specification, but the cost structure may change over time or the increased costs may be deemed a suitable tradeoff in some embodiments. Accordingly, this may be interesting since a “swelling” type piezoelectric transducer may be more easily mechanically coupled to the tactile region in the tactile frequency band than the “bending” type in some embodiments. In the case of a bending piezoelectric transducer, the typical maximum displacement at the tip (cantilevered free end) can be of above 0.1 mm, such as between 0.2 and 0.7 mm, for instance. In the case of a swelling piezoelectric transducer, the change in thickness can be in the order of 1 μm, or even in the order of 0.1 μm, e.g. between 0.1 and 1 μm, for instance.

Overall, in some embodiments, it can be desired for the piezoelectric transducer to generate a vibratory stimulation ranging from 3 to 1000 Hz, with a magnitude reaching the order of magnitude of 1 N, such as a typical value around 0.01 N peak value and acceleration reaching 3-4 m/s².

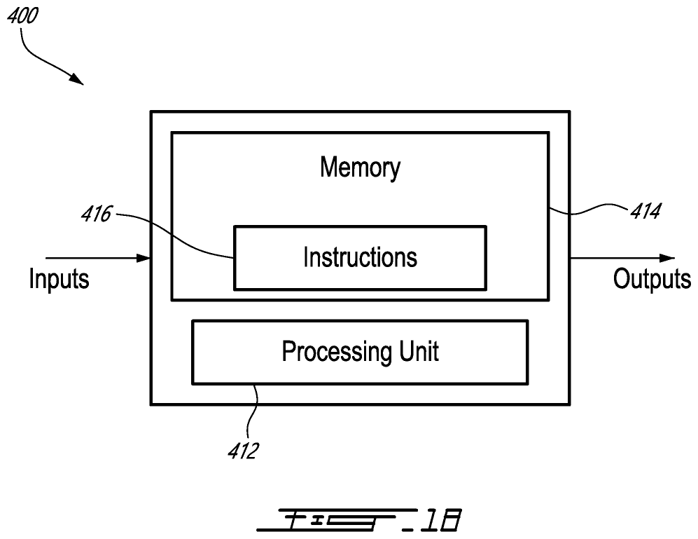

Referring to FIG. 18, it will be understood that the expression “computer” 400 as used herein is not to be interpreted in a limiting manner. It is rather used in a broad sense to generally refer to the combination of some form of one or more processing units 412 and some form of memory system 414 accessible by the processing unit(s). The memory system can be of the non-transitory type. The use of the expression “computer” in its singular form as used herein includes within its scope the combination of a two or more computers working collaboratively to perform a given function. Moreover, the expression “computer” as used herein includes within its scope the use of partial capabilities of a given processing unit.

A processing unit can be embodied in the form of a general-purpose micro-processor or microcontroller, a digital signal processing (DSP) processor, an integrated circuit, a field programmable gate array (FPGA), a reconfigurable processor, and a programmable read-only memory (PROM, to name a few examples.

The memory system can include a suitable combination of any suitable type of computer-readable memory located either internally, externally, and accessible by the processor in a wired or wireless manner, either directly or over a network such as the Internet. A computer-readable memory can be embodied in the form of random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) to name a few examples.

A computer can have one or more input/output (I/O) interface to allow communication with a human user and/or with another computer via an associated input, output, or input/output device such as a keyboard, a mouse, a touchscreen, an antenna, a port, etc. Each I/O interface can enable the computer to communicate and/or exchange data with other components, to access and connect to network resources, to serve applications, and/or perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, Bluetooth, WiMAX), SS7 signaling network, fixed line, local area network, wide area network, to name a few examples.

It will be understood that a computer can perform functions or processes via hardware or a combination of both hardware and software. For example, hardware can include logic gates included as part of a silicon chip of a processor. Software (e.g. application, process) can be in the form of data such as computer-readable instructions stored in a non-transitory computer-readable memory accessible by one or more processing units. With respect to a computer or a processing unit, the expression “configured to” relates to the presence of hardware or a combination of hardware and software which is operable to perform the associated functions. Different elements of a computer, such as processor and/or memory, can be local, or in part or in whole remote and/or distributed and/or virtual. The methods and systems of the present disclosure may be implemented in a high level procedural or object oriented programming or scripting language, or a combination thereof, to communicate with or assist in the operation of a computer system, for example the controller 31. Alternatively, the methods and systems described herein may be implemented in assembly or machine language. The language may be a compiled or interpreted language. Program code for implementing the methods and systems described herein may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems described herein may also be considered to be implemented by way of a non-transitory computer-readable storage medium having a computer program stored thereon. The computer program may comprise computer-readable instructions which cause a computer, or more specifically the processing unit 402 of the computing device 400, to operate in a specific and predefined manner to perform the functions described herein, for example those described in the method 500.

Computer-executable instructions may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments. The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

As can be understood, the examples described above and illustrated are intended to be exemplary only. The scope is indicated by the appended claims.

1. A system for imparting tactile sensations to different tactile regions of a hand of a user, the system comprising-:
a garment configured to be worn by a hand of a user; and
a plurality of piezoelectric transducers secured to the garment at different locations of the garment, the plurality of piezoelectric transducers being mechanically coupled to the different tactile regions of the hand of the user when the garment is worn by the hand of the user, each piezoelectric transducer being drivable to impart tactile sensations to a corresponding one of the tactile regions by a signal having at least one of intensity, frequency and temporal acoustic traits.

2. The system of claim 1 wherein, when the garment is worn by the hand of the user, the garment applies a spring constant of between 85 and 350 N/m in mechanically coupling one or more of the piezoelectric transducers to corresponding tactile regions of the hand of the user.

3. The system of claim 1, 2 wherein the piezoelectric transducers are of the bending type.

4. The system of claim 3 wherein the garment mechanically couples a proximal end of at least one of the piezoelectric transducers to a corresponding tactile region, a distal end of the at least one of the piezoelectric transducers being free, the at least one of the piezoelectric transducers being cantilevered by the garment between the proximal end and the distal end.

5. The system of claim 1 wherein a mass is secured to the at least one of the piezoelectric transducers.

6. The system of claim 5 wherein the mass is of between 1 g and 3.5 g.

7. The system of claim 6 wherein the mass is secured to the piezoelectric transducer between the proximal end and the distal end.

8. The system of claim 1 wherein a supplementary piezoelectric transducer is mechanically coupled to the at least one piezoelectric transducer to cover a different portion of the tactile frequency band than the at least one piezoelectric transducer.

9. The system of claim 1 wherein the piezoelectric transducers are of the swelling type.

10. The system of claim 1 wherein the garment includes a hook and loop fastener arrangement configured for tightening at least one of the piezoelectric transducer against a corresponding tactile region, the hook and loop fastener arrangement being made of nylon.

11. The system of claim 1 wherein at least one of the piezoelectric transducers is adhered to the garment using a cyanoacrylate adhesive.

12. The system of claim 1 wherein the different tactile regions of the hand include at least portions of two fingers of the hand.

13. The system of claim 1 wherein the different tactile regions of the hand include at least a portion of a finger and a portion of a palm of the hand.

14. The system of claim 1 wherein the different tactile regions of the hand includes portions of five fingers of the hand and a portion of a palm of the hand.

15. A method of imparting tactile sensations, the method comprising-:
providing a plurality of transducers mechanically coupled to at least one hand of a user, different ones of the plurality of transducers being mechanically coupled to different tactile regions of the at least one hand of the user;
driving a first one of the plurality of transducers via a first channel signal to impart tactile sensations to a first one of the tactile regions, the first channel signal having at least one of intensity, frequency, and temporal acoustic traits; and
driving a second one of the plurality of transducers via a second channel signal to impart tactile sensations to a second one of the tactile regions, the second channel signal having at least one of intensity, frequency and temporal acoustic traits; the acoustic traits of the second channel signal being different from the acoustic traits of the first channel signal.

16. The method of claim 15 further comprising:
driving a third one of the plurality of transducers via a third channel signal to impart tactile sensations to a third one of the tactile regions, the third channel signal having at least one of intensity, frequency, and temporal acoustic traits; and
driving a fourth one of the plurality of transducers via a fourth channel signal to impart tactile sensations to a fourth one of the tactile regions, the fourth channel signal having at least one of intensity, frequency and temporal acoustic traits; the acoustic traits of each of the first channel, second channel, third channel and fourth channel being different from the acoustic traits of the other ones of the first channel, second channel, third channel and fourth channel.

17. The method of claim 15 further comprising driving a plurality of additional ones of the plurality of transducers via respective additional channel signals to impart tactile sensations to respective ones of the tactile regions, each additional channel signals having at least one of intensity, frequency, and temporal acoustic traits, the acoustic traits of each of the first channel signal, the second channel signal, and the additional channel signals being different from the acoustic traits of other ones of the channel signals.

18. The method of claim 17 wherein the plurality of transducers includes at least 8 transducers and the additional channel signals include at least 6 additional channel signals.

19. The method of claim 17 wherein the plurality of transducers includes at least 12 transducers and the additional channel signals include at least 10 additional channel signals.

20. The method of claim 15 wherein said different tactile regions of at least one hand of the user includes at least a first tactile region on a first finger of a first hand and a second tactile region on a second finger of the first hand.

21.-36. (canceled)
